SEQUENCE LISTING (17 sequences)

SEQ ID NO: 1 - protein, 226 aa, Artificial Sequence
  Synthesized
MKIEEGKLTNPGVSAWQVNTAYTAGQLVTYNGKTYKCLQPHTSLAGWEPSNVPALWQLQNNGNNGLELRESGAISGDSLISLASTGKRVSIKDLLDEKDFEIWAINEQTMKLESAKVSRVFCTGKKLVYILKTRLGRTIKATANHRFLTIDGWKRLDELSLKEHIALPRKLESSSLQLSPEIEKLSQSDIYWDSIVSITETGVEEVFDLTVPGPHNFVANDIIVHN

SEQ ID NO: 2 - protein, 6 aa, Artificial Sequence
  Synthesized
ENLYFQ

SEQ ID NO: 3 - protein, 10 aa, Artificial Sequence
  Synthesized
PGGGGSGGGS

SEQ ID NO: 4 - protein, 34 aa, Artificial Sequence
  Synthesized
SVSEIQLMHNLGKHLNSMERVEWLRKKLQDVHNF

SEQ ID NO: 5 - protein, 30 aa, Artificial Sequence
  Synthesized
HAEGTFTSDVSYLEGQAAKEFLAWLVRGRG

SEQ ID NO: 6 - protein, 39 aa, Artificial Sequence
  Synthesized
HGEGTFTSDLSKQMEEEAVRLFIEWLKNGGPSSGAPPPS

SEQ ID NO: 7 - protein, 277 aa, Artificial Sequence
  Synthesized
MKIEEGKLTNPGVSAWQVNTAYTAGQLVTYNGKTYKCLQPHTSLAGWEPSNVPALWQLQNNGNNGLELRESGAISGDSLISLASTGKRVSIKDLLDEKDFEIWAINEQTMKLESAKVSRVFCTGKKLVYILKTRLGRTIKATANHRFLTIDGWKRLDELSLKEHIALPRKLESSSLQLSPEIEKLSQSDIYWDSIVSITETGVEEVFDLTVPGPHNFVANDIIVHNPGGGGSGGGGSENLYFQSVSEIQLMHNLGKHLNSMERVEWLRKKLQDVHNF

SEQ ID NO: 8 - protein, 274 aa, Artificial Sequence
  Synthesized
MKIEEGKLTNPGVSAWQVNTAYTAGQLVTYNGKTYKCLQPHTSLAGWEPSNVPALWQLQNNGNNGLELRESGAISGDSLISLASTGKRVSIKDLLDEKDFEIWAINEQTMKLESAKVSRVFCTGKKLVYILKTRLGRTIKATANHRFLTIDGWKRLDELSLKEHIALPRKLESSSLQLSPEIEKLSQSDIYWDSIVSITETGVEEVFDLTVPGPHNFVANDIIVHNPGGGGSGGGGSENLYFQHAEGTFTSDVSSYLEGQAAKEFIAWLVRGRG

SEQ ID NO: 9 - protein, 260 aa, Artificial Sequence
  Synthesized
MKIEEGKLTNPGVSAWQVNTAYTAGQLVTYNGKTYKCLQPHTSLAGWEPSNVPALWQLQNNGNNGLELRESGAISGDSLISLASTGKRVSIKDLLDEKDFEIWAINEQTMKLESAKVSRVFCTGKKLVYILKTRLGRTIKATANHRFLTIDGWKRLDELSLKEHIALPRKLESSSLQLSPEIEKLSQSDIYWDSIVSITETGVEEVFDLTVPGPHNFVANDIIVHNSVSEIQLMHNLGKHLNSMERVEWLRKKLQDVHNF

SEQ ID NO: 10 - DNA, 35 bp, Artificial Sequence
  syntesized
ttccaagctcttctaacagcgtgagcgagatccag

SEQ ID NO: 11 - DNA, 42 bp, Artificial Sequence
  syntesized
ccaattctgcagttagaagttgtgcacgtcttgcagcttctt

SEQ ID NO: 12 - DNA, 62 bp, Artificial Sequence
  Synthesized
ttccaagctcttctaacccgggtggcggtggctctggtggaggtgggtctgaaaacctgtac

SEQ ID NO: 13 - DNA, 42 bp, Artificial Sequence; identical to SEQ ID NO: 11
  Synthesized

SEQ ID NO: 14 - DNA, 89 bp, Artificial Sequence
  Synthesized
ttccaagctcttctaacccgggtggcggtggctctggtggaggtgggtctgaattcgaaaacctgtacttccagcacgctgagggtact

SEQ ID NO: 15 - protein, 36 aa, Artificial Sequence
  Synthesized
CCAATTCTGCAGTTAACCGCGACCGCGAACCAGCCA

SEQ ID NO: 16 - protein, 191 aa, Artificial Sequence [sequence omitted]

SEQ ID NO: 17 - protein, 202 aa, Artificial Sequence
  Synthesized
CITGDALVALPEGESVRIADIVPGARPNSDNAIDLKVLDRHGNPVLADRLFHSGEHPVYTVRTVEGLRVTGTANHPLLCLVDVAGVPTLLWKLIDEIKPGDYAVIQRSAFSVDCAGFARGKPEFAPTTYTVGVPGLVRFLEAHHRDPDAQAIADELTDGRFYYAKVASVTDAGVQPVYSLRVDTADHAFITNGFVSHATGLT

(19) United States
(12) Patent Application Publication
(10) Pub. No.: US 2019/0352365 A1
(43) Pub. Date: Nov. 21, 2019

(54) EXPRESSION CONSTRUCT AND METHOD FOR PRODUCING PROTEINS OF INTEREST

(71) Applicant: Savior Lifetec Corporation, Miaoli County (TW)

(72) Inventors: Shu-Ping YANG, Miaoli County (TW); Hong-Zhang WANG, Miaoli County (TW); Chi-Chao HU, Miaoli County (TW); Jia-Hau SHIU, Miaoli County (TW); Shih-Hsie PAN, Princeton, NJ (US); Mannching Sherry KU, Burbank, CA (US)

(73) Assignee: Savior Lifetec Corporation, Miaoli County (TW)

(21) Appl. No.: 16/476,294

(22) PCT Filed: Jan. 18, 2018
(86) PCT No.: PCT/US18/14124

§ 371 Date: Jul. 7, 2019

Related U.S. Application Data
(60) Provisional application No. 62/447,452, filed on Jan. 18, 2017.

(51) Int. Cl. C07K 14/605; C07K 14/635; C07K 14/61; C07K 14/62; C07K 14/655; C12P 21/02

(52) U.S. Cl. CPC C07K 14/605; C07K 14/635; C07K 14/61; C07K 2319/50; C07K 14/655; C12P 21/02; C07K 2319/20; C07K 14/62

(57) ABSTRACT

Disclosed herein are expression constructs and methods for producing a protein of interest. According to some examples, an expression construct includes a nucleotide sequence encoding a fusion protein. To produce the protein of interest, the expression construct is transformed into a host cell, which is cultured under conditions that allow the expression of the fusion protein. The fusion protein has at least one expression unit; each expression unit has, sequentially, an affinity tag moiety, a spacer moiety, an enzymatic cleavage site, and a protein of interest moiety. The fusion protein is isolated from the host cell, solubilized, and refolded. The refolded fusion protein is cleaved to release the protein of interest.

### CROSS REFERENCE TO RELATED APPLICATION

This application claims priority from U.S. provisional patent application No. 62/447,452, filed Jan. 18, 2017, which is incorporated herein by reference.

### BACKGROUND OF THE INVENTION

### 1. Field of the Invention

The present disclosure relates to the manufacture of proteins of interest; more particularly, to proteins of interest for use as therapeutic peptides.

### 2. Description of Related Art

Therapeutic proteins or peptides accounts for the most dominant segment of currently marketed biological products (also known as biologics). Generally, therapeutic proteins include antibody-based drugs, Fc fusion proteins, growth factors, hormones, interferons, interleukins, anticoagulants, blood factors, engineered protein scaffolds, and thrombolytic. Since the approval of human insulin in 1982, more than one hundred recombinant therapeutic proteins have been approved for clinical use in the United States of America and European Union.

Unlike small-molecule drugs, which are chemically synthesized and their structure is known, most therapeutic proteins, as well as other biologics, are complex mixtures that are not easily identified or characterized. Biological products, including those manufactured by recombinant technology, tend to be heat sensitive and susceptible to microbial contamination, and as such are manufactured under conditions that are more stringent than those used in the manufacture of small-molecule drugs.

The manufacture of therapeutic proteins is a multi-step process involving the manufacture processes of bulk drug substance and drug products. Generally, the protein products are first produced in microbial cells (e.g., prokaryotic) using expression constructs. The thus-produced proteins can be produced in prokaryotic cells, using large scale protein production schemes such as fermentation and cell culture. The thus-produced protein products are then purified, buffer exchanged, and stored as the bulk drug substances. The bulk drug substance may be filled directly, diluted, or compounded with buffer and excipient(s) to make a final pharmaceutical formulation, which is then filled or packaged into suitable containers and becomes the drug product.

A follow-on biologic, also known as a biosimilar, is a product approved based on a showing that it is highly similar to an FDA-approved biological product (i.e., a reference product), and has no clinically meaningful differences in terms of safety and effectiveness from the reference product.

The demand for therapeutic proteins has led to a number of innovations in the manufacture process thereof. However, for the manufacture of follow-on biologics, there is little room left to improve for only minor differences in clinically inactive components are allowable in biosimilar products. For example, the type of expression system will affect the types of process- and product-related substances, impurities, and contaminants (including potential adventitious agents) that may be present in the protein product. Accordingly, minimizing the differences between the expression systems used for manufacturing the reference products and the biosimilar to the extent possible is often critical in the production of the biosimilar product.

In view of the foregoing, there exists a need in the related art for providing methods for producing proteins of interest, such as a protein drug that is highly similar to an FDA-approved biological product.

### SUMMARY

The following presents a simplified summary of the disclosure in order to provide a basic understanding to the reader. This summary is not an extensive overview of the disclosure and it does not identify key/critical elements of the present invention or delineate the scope of the present invention. Its sole purpose is to present some concepts disclosed herein in a simplified form as a prelude to the more detailed description that is presented later.

In one aspect, the present disclosure is directed to a novel expression construct for preparing a protein of interest. Specifically, the present expression construct adopts a novel spacer moiety. The present expression construct is advantageous in that it provides an alternative to the expression construct currently used by other manufactures, while yielding the protein of interest that is highly similar to the existing products.

According to some embodiments of the present disclosure, the expression construct comprises a nucleotide sequence encoding a fusion protein, wherein the fusion protein comprises, sequentially, an affinity tag moiety, a spacer moiety, an enzymatic cleavage site, and a protein of interest moiety.

In some embodiments, the expression construct further comprises a promoter upstream to the nucleotide sequence encoding the fusion protein.

According to certain embodiments, the spacer moiety comprises the amino acid sequence of SEQ ID NO: 2. In some embodiments, the spacer moiety consists of the amino acid sequence of SEQ ID NO: 2.

According to optional embodiments, the protein of interest comprises the amino acid sequence of SEQ ID NO: 4, 5, 6, or 16. For example, the molecule of interest is selected from the group consisting of SEQ ID NOs: 4, 5, 6, and 16.

According to certain embodiments, the affinity tag moiety is a chitin binding domain (CBD)-intein. For example, the CBD-intein comprises the amino acid sequence of SEQ ID NO: 1. In some embodiments, the CBD-intein consists of the amino acid sequence of SEQ ID NO: 1.

According to some embodiments, the enzymatic cleavage site is a tobacco etch virus (TEV) cleavage recognition site. For example, the TEV cleavage recognition site comprises the amino acid sequence of SEQ ID NO: 3. In some embodiments, the TEV cleavage recognition site consists of the amino acid sequence of SEQ ID NO: 3.

According to some embodiments, the fusion protein comprises the amino acid sequence of SEQ ID NO: 7 or 8.

In another aspect, the present disclosure is directed to an expression system for preparing a protein of interest.

According to certain embodiments of the present disclosure, the expression system comprises a host cell and an expression construct that comprises a nucleotide sequence encoding a fusion protein, wherein the fusion protein comprises, sequentially, an affinity tag moiety, a spacer moiety, an enzymatic cleavage site, and a protein of interest moiety.

According to certain embodiments, the spacer moiety comprises the amino acid sequence of SEQ ID NO: 2. In some embodiments, the spacer moiety consists of the amino acid sequence of SEQ ID NO: 2.

According to optional embodiments, the protein of interest comprises the amino acid sequence of SEQ ID NO: 4, 5, 6, or 16. For example, the protein of interest is selected from the group consisting of SEQ ID NOs: 4, 5, 6, and 16.

According to certain embodiments, the affinity tag moiety is a chitin binding domain (CBD)-intein. For example, the CBD-intein comprises the amino acid sequence of SEQ ID NO: 1. In some embodiments, the CBD-intein consists of the amino acid sequence of SEQ ID NO: 1.

According to some embodiments, the enzymatic cleavage site is a tobacco etch virus (TEV) cleavage recognition site. For example, the TEV cleavage recognition site comprises the amino acid sequence of SEQ ID NO: 3. In some embodiments, the TEV cleavage recognition site consists of the amino acid sequence of SEQ ID NO: 3.

According to some embodiments, the fusion protein comprises the amino acid sequence of SEQ ID NO: 7 or 8.

According to some embodiments, the host cell is a prokaryotic cell, such as E. coli cells.

In another aspect, the present disclosure is directed to a novel expression construct capable of producing multiple copies of a protein of interest. Specifically, the present expression construct adopts a novel spacer moiety. The present expression construct is further advantageous in that it substantially increased the yield of the protein of interest. Further, by using the novel vector design scheme according to the above-mentioned aspect of the present disclosure, the protein of interest prepared using this vector is highly similar to the existing products.

According to some embodiments of the present disclosure, the expression construct comprises two or more expression units, in which each expression unit comprises a nucleotide sequence encoding a fusion protein, wherein the fusion protein comprises, sequentially, an affinity tag moiety, a spacer moiety, an enzymatic cleavage site, and a protein of interest moiety.

In some embodiments, each expression unit further comprises a promoter upstream to the nucleotide sequence encoding the fusion protein.

According to certain embodiments, the spacer moiety comprises the amino acid sequence of SEQ ID NO: 2. In some embodiments, the spacer moiety consists of the amino acid sequence of SEQ ID NO: 2.

According to optional embodiments, the protein of interest comprises the amino acid sequence of SEQ ID NO: 4, 5, 6, or 16. For example, the molecule of interest is selected from the group consisting of SEQ ID NOs: 4, 5, 6, and 16.

According to certain embodiments, the affinity tag moiety is a chitin binding domain (CBD)-intein. For example, the CBD-intein comprises the amino acid sequence of SEQ ID NO: 1. In some embodiments, the CBD-intein consists of the amino acid sequence of SEQ ID NO: 1.

According to some embodiments, the enzymatic cleavage site is a tobacco etch virus (TEV) cleavage recognition site. For example, the TEV cleavage recognition site comprises the amino acid sequence of SEQ ID NO: 3. In some embodiments, the TEV cleavage recognition site consists of the amino acid sequence of SEQ ID NO: 3.

According to some embodiments, the fusion protein comprises the amino acid sequence of SEQ ID NO: 7 or 8.

In another aspect, the present disclosure is directed to an expression system for preparing a protein of interest.

According to certain embodiments of the present disclosure, the expression system comprises a host cell and an expression construct that comprises a nucleotide sequence encoding a fusion protein, wherein the fusion protein comprises, sequentially, an affinity tag moiety, a spacer moiety, an enzymatic cleavage site, and a protein of interest moiety.

According to certain embodiments, the spacer moiety comprises the amino acid sequence of SEQ ID NO: 2. In some embodiments, the spacer moiety consists of the amino acid sequence of SEQ ID NO: 2.

According to optional embodiments, the protein of interest comprises the amino acid sequence of SEQ ID NO: 4, 5, 6, or 16. For example, the protein of interest is selected from the group consisting of SEQ ID NOs: 4, 5, 6, and 16.

According to certain embodiments, the affinity tag moiety is a chitin binding domain (CBD)-intein. For example, the CBD-intein comprises the amino acid sequence of SEQ ID NO: 1. In some embodiments, the CBD-intein consists of the amino acid sequence of SEQ ID NO: 1.

According to some embodiments, the enzymatic cleavage site is a tobacco etch virus (TEV) cleavage recognition site. For example, the TEV cleavage recognition site comprises the amino acid sequence of SEQ ID NO: 3. In some embodiments, the TEV cleavage recognition site consists of the amino acid sequence of SEQ ID NO: 3.

According to some embodiments, the fusion protein comprises the amino acid sequence of SEQ ID NO: 7 or 8.

According to some embodiments, the host cell is a prokaryotic cell, such as E. coli cells.

In still another aspect, the present disclosure is directed to methods for producing a protein of interest. In particular, the present method uses the novel expression construct according to embodiments of the above-mentioned aspects of the present disclosure. Accordingly, embodiments of the present disclosure provide manufacturing methods that are alternative to the processes used by other manufacturers, while maintaining the similarity between the protein of interest and the product from another source. For example, in the case where the protein of interest is a protein, the protein prepared by the present method and a reference product are highly similar in terms of the primary, secondary, tertiary and/or quaternary structures, post-translational modifications, and functional activities.

According to one embodiment of the present disclosure, the method comprises the steps of (a) providing an expression construct according to any embodiments of the above-mentioned aspects; (b) transforming a host cell with the expression construct and culturing the host cell under conditions that allow the expression of the fusion protein; (c) lysing the host cell to obtain a lysate comprising a soluble fraction and an insoluble fraction; (d) optionally purifying the insoluble fraction to obtain a purified insoluble fraction; (e) solubilizing the insoluble fraction from the step (c) or the purified insoluble fraction from the step (d) to obtain a solubilized fusion protein; (f) suspending the solubilized fusion protein in a renaturation buffer thereby allowing the refolding of the solubilized fusion protein to obtain a refolded fusion protein; and

(g) cleaving the enzymatic cleavage site of the refolded fusion protein to release the protein of interest; and (g) cleaving the enzymatic cleavage site of the refolded fusion protein to release the protein of interest.

According to some embodiments, the step (d) is performed using an affinity column that is specific to the affinity tag moiety, such as a chitin column.

According to some embodiments, the host cell is a prokaryotic cell, such as E. coli. cells.

In yet another aspect, the present disclosure is directed to an expression vector having multiple copies of a protein of interest. This expression vector can be used to manufacture a protein of interest with an increased yield.

According to embodiments of the present disclosure, the expression construct comprises an expression unit, wherein each expression unit comprises a nucleotide sequence encoding a fusion protein that comprises two protein of interest moieties, and a self-cleavage peptide or catalytic cleavage protein inserted between said two protein of interest moieties.

According to some optional embodiments of the present disclosure, the self-cleavage peptide or catalytic cleavage protein comprises the amino acid sequence of SEQ ID NO: 17.

According to optional embodiments, the protein of interest comprises the amino acid sequence of SEQ ID NO: 4, 5, 6, or 16. For example, the protein of interest is selected from the group consisting of SEQ ID NOs: 4, 5, 6, and 16.

In optional embodiments, each expression unit further comprises a promoter upstream to the nucleotide sequence encoding the fusion protein.

According to some embodiments of the present disclosure, the expression construct comprises two expression constructs.

Many of the attendant features and advantages of the present disclosure will becomes better understood with reference to the following detailed description considered in connection with the accompanying drawings.

### BRIEF DESCRIPTION OF THE DRAWINGS

The present description will be better understood from the following detailed description read in light of the accompanying drawings, where:

FIGS. 1A to 1C provide photographs showing the intein-mediated self-cleavage of a fusion protein, according to Example 1 of the present disclosure;

FIGS. 2A and 2B provide photographs showing the TEV protease-mediated cleavage of a fusion protein, according to Example 2 of the present disclosure;

FIG. 3 is the HPLC elution profile of the target protein according to Example 2 of the present disclosure;

FIG. 4 provides photographs showing the TEV protease-mediated cleavage of a fusion protein, according to Example 3 of the present disclosure;

FIG. 5 is the HPLC elution profile of the target protein according to Example 3 of the present disclosure;

FIG. 6 is a diagram illustrating the experimental design according to Example 4 of the present disclosure;

FIG. 7A to FIG. 7D provide photographs showing the results of Example 4 of the present disclosure;

FIG. 8A to FIG. 8C provide photographs showing the results of Example 4 of the present disclosure;

FIG. 9 is a diagram illustrating the experimental design according to Example 4 of the present disclosure;

FIG. 10A and FIG. 10B provide photographs showing the results of Example 4 of the present disclosure;

FIG. 11A to FIG. 11D show the results of Example 4 of the present disclosure;

FIG. 12 is a diagram illustrating the experimental design according to Example 5 of the present disclosure; and

FIG. 13A and FIG. 13B provide photographs showing the results of Example 5 of the present disclosure.

### DESCRIPTION

The detailed description provided below in connection with the appended drawings is intended as a description of the present examples and is not intended to represent the only forms in which the present example may be constructed or utilized. The description sets forth the functions of the example and the amino acid sequence of steps for constructing and operating the example. However, the same or equivalent functions and sequences may be accomplished by different examples.

For convenience, certain terms employed in the specification, examples and appended claims are collected here. Unless otherwise defined herein, scientific and technical terminologies employed in the present disclosure shall have the meanings that are commonly understood and used by one of ordinary skill in the art.

Unless otherwise required by context, it will be understood that singular terms shall include plural forms of the same and plural terms shall include the singular. Also, as used herein and in the claims, the terms “at least one” and “one or more” have the same meaning and include one, two, three, or more. Furthermore, the phrases “at least one of A, B, and C”, “at least one of A, B, or C” and “at least one of A, B and/or C,” as use throughout this specification and the appended claims, are intended to cover A alone, B alone, C alone, A and B together, B and C together, A and C together, as well as A, B, and C together.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in the respective testing measurements. Also, as used herein, the term “about” generally means within 10%, 5%, 1%, or 0.5% of a given value or range. Alternatively, the term “about” means within an acceptable standard error of the mean when considered by one of ordinary skill in the art. Other than in the operating/working examples, or unless otherwise expressly specified, all of the numerical ranges, amounts, values and percentages such as those for quantities of materials, durations of times, temperatures, operating conditions, ratios of amounts, and the likes thereof disclosed herein should be understood as modified in all instances by the term “about.” Accordingly, unless indicated to the contrary, the numerical parameters set forth in the present disclosure and attached claims are approximations that can vary as desired. At the very least, each numerical parameter should at least be construed in light of the number of reported significant digits and by applying ordinary rounding techniques. Ranges can be expressed herein as from one endpoint to another endpoint or between two endpoints. All ranges disclosed herein are inclusive of the endpoints, unless specified otherwise.

For all the nucleotide and amino acid sequences disclosed herein, it is understood that equivalent nucleotides and amino acids can be substituted into the amino acid sequences without affecting the function of the amino acid sequences. Such substitution is within the ability of a person of ordinary skill in the art.

Throughout the present disclosure, the term “protein” is intended to include the amino acid sequence of a full-length native protein, or a fragment thereof, subject to those modifications that do not significantly change the specific properties of the native protein.

As used herein, the term “expression construct” refers to nucleic acid sequences containing a desired coding sequence and appropriate nucleic acid sequences necessary for the expression of the operably linked coding sequence in a particular host organism. Nucleic acid sequences necessary for expression in prokaryotic expression systems are known to persons having ordinary skill in the art.

The term “enzymatic cleavage site” denotes a defined amino acid sequence that allows cleavage of a protein or peptide containing this sequence by a selective protease.

The term “fusion protein” as used herein refers to a hybrid polypeptide that comprises protein domains from at least two different proteins. The term “protein of interest” as used herein refers to the peptide whose expression is desired within the hybrid polypeptide. As used herein, the term “affinity tag moiety” refers to a peptide enabling a specific interaction with a specific ligand.

As used herein, the term “host cell” is intended to include any cellular system that can be used to express the fusion proteins of the present disclosure or fragments thereof. Suitable host cells include prokaryotic microorganisms (such as E. coli).

The invention disclosed herein focuses on the manufacture of a protein of interest. To this end, a novel expression construct is provided by the present disclosure. As could be appreciated, an expression system comprising the above-mentioned expression construct also falls within the scope of the present disclosure. Furthermore, some embodiments of the present disclosure provide manufacturing methods that are alternative to the processes used by other manufacturers, while maintaining the similarity between the protein of interest and the product from other source. For example, the protein of interest can be a protein and in these cases, the protein produced by the present method may be highly similar to a reference protein in terms of the primary, secondary, tertiary and/or quaternary structures, post-translational modifications, and functional activities.

According to embodiments of the present disclosure, the method comprises the steps discussed below. The expression construct and expression system for use in the present preparation methods are also discussed herein.

First, an expression construct that comprises at least one expression unit is provided. Each expression unit comprises a nucleotide sequence encoding a fusion protein. Briefly, the nucleotide sequence encoding the fusion protein or a portion thereof is first synthesized, which is then amplified using primers that are specifically designed to incorporate restriction sites for subsequent cloning step. Next, the amplified nucleotide is cloned into an expression vector under the control of a suitable promoter. The choice of expression vector is dependent upon the choice of host cell, and may be selected to have the desired expression and post-translational characteristics in the selected host cell. For example, in some cases, the host cell is E. coli, and the expression vector is a pTWIN vector.

According to embodiments of the present disclosure, the fusion protein comprises, sequentially, an affinity tag moiety, a spacer moiety, an enzymatic cleavage site, and a protein of interest moiety.

In the case where the expression construct comprises multiple expression units, each expression unit has a promoter upstream to the nucleotide sequence encoding the fusion protein.

The affinity tag moiety is incorporated to allow purification of the interested proteins out from other endogenous proteins that are expressed by the host cell. According to certain embodiments, the affinity tag moiety is a chitin binding domain (CBD)-intein. However, the present disclosure is not limited thereto; rather, it is conceivable that other purification tags, such as his-tag, can be used in lieu of CBD to mediate the subsequent affinity purification. According to embodiments of the present disclosure, the CBD-intein comprises the amino acid sequence of SEQ ID NO: 1. In some embodiments, the CBD-intein consists of the amino acid sequence of SEQ ID NO: 1.

The spacer moiety is a stretch of amino acid residues between the affinity tag moiety and the enzymatic cleavage site. According to certain embodiments of the present invention, the spacer moiety comprises a proline (P) residue followed by a flexible peptide consisting of glycine (G) and serine (S) residues. For example, the spacer moiety may have the amino acid sequence of P(GGGGS)₂ (SEQ ID NO: 2). According to embodiments of the present disclosure, the incorporation of the spacer moiety improves the cleavage efficiency of the subsequent enzymatic cleavage step.

The enzymatic cleavage site allows the enzymatic cleavage of the fusion protein so that the protein of interest could be freed from the rest of the fusion protein. According to some embodiments, the enzymatic cleavage site is a tobacco etch virus (TEV) cleavage recognition site. TEV protease recognizes a linear epitope of the general form E-Xaa-Xaa-Y-Xaa-Q-(G/S), with cleavage occurring between Q and G or Q and S. According to embodiments of the present disclosure, the TEV cleavage recognition site comprises the amino acid sequence of ENLYFQ (SEQ ID NO: 3). In some embodiments, the TEV cleavage recognition site consists of the amino acid sequence of SEQ ID NO: 3. As could be appreciated, in some embodiments, the protein of interest is a protein, in which he first amino acid residue of the protein is serine or glycine, and accordingly, the cleavage would occur between the last residue of the TEV cleavage recognition site and the first residue of the protein. In some cases, the first amino acid residue of the protein is neither serine nor glycine; yet, a cleavage between the last residue of the TEV cleavage recognition site and the first residue of the protein still occurs.

According to embodiments of the present disclosure, the protein of interest could be any protein that is desired. In some cases, the protein is a therapeutic protein. More particularly, the protein thus prepared could be used as the active component of a biosimilar product. To name a few, the protein of interest could be a recombinant parathyroid hormone (e.g., teriparatide) or a glucagon-like peptide-1 receptor agonist (liraglutide). For example, the protein produced by the present method comprises the amino acid sequence of SEQ ID NO: 4, 5, 6, or 16. In some examples, the protein is selected from the group consisting of SEQ ID NOs: 4, 5, 6, and 16.

To generate an expression system according to embodiments of the present disclosure, the expression vector is then introduced into a host cell by various methods known in the art. According to some embodiments, the host cell is a prokaryotic cell, such as E. coli cells. Once the expression vector has been introduced into an appropriate host cell, the host cell may be cultured under conditions that allow the expression of the fusion protein. For example, the host cells are cultivated under defined media and temperature conditions. The medium may be a nutrient, minimal, selective, differential, or enriched medium. Growth and expression temperature of the host cell may range from 4° C. to 45° C.; preferably, from 30° C. to 39° C.

The fusion protein is then isolated from the host cells. According to some embodiments, the host cells are lysed to obtain a lysate comprising a soluble fraction and an insoluble fraction. In some optional embodiments, the insoluble fraction is further purified using an affinity column that is specific to the affinity tag moiety to obtain a purified insoluble fraction. As could be appreciated, the present method is not limited to the purification methods mentioned above; rather, any other suitable purification techniques can be used in the present method.

Next, the insoluble fraction or the purified insoluble fraction from the step is solubilized to obtain a solubilized fusion protein. For example, the insoluble fraction or the purified insoluble fraction is treated with a denaturing solution (e.g., guanidine hydrochloride or urea) to denature and solubilize the fusion protein comprised in the fraction.

Then, in the refolding step, the solubilized fusion protein is suspended in a renaturation buffer by dilution or dialysis in order to allow the fusion protein to obtain its native, biologically active conformation. Optionally, the refolded fusion protein is separated from other aberrantly folded products by concentration and chromatography.

Thereafter, the refolded fusion protein is cleaved with a protease at the enzymatic cleavage site thereof to release the protein of interest.

In another aspect, the present invention is directed to an expression vector comprising multiple copies of a protein of interest moiety.

According to embodiments of the present disclosure, the expression construct comprises an expression unit that comprises a nucleotide sequence encoding a fusion protein, wherein the fusion protein comprises two protein of interest moieties, and a self-cleavage peptide or catalytic cleavage protein inserted between said two protein of interest moieties.

In this case, each expression unit may further comprise a promoter upstream to the nucleotide sequence encoding the fusion protein. In other word, two copies of a protein of interest moiety are under the control of a single promoter. In the case where the expression construct comprises two or more expression units, each expression unit may comprise its own promoter.

By selecting a proper self-cleavage peptide or catalytic cleavage protein, the multiple copies of the protein of interest moiety prepared using this expression vector may have the same amino acid sequence. This feature is of particular advantages to the manufacture of proteins for use in a drug product, for a minor difference in the amino acid sequence of the proteins may deteriorate the therapeutic effect of the drug product significantly.

For example, the catalytic cleavage protein may be a thiol-induced cleavage protein. According to some optional embodiments of the present disclosure, the self-cleavage peptide or catalytic cleavage protein comprises the amino acid sequence of SEQ ID NO: 17.

According to certain embodiments of the present disclosure, each protein of interest moiety is selected from the group consisting of SEQ ID NO: 4, 5, 6, and 16.

The following Examples are provided to elucidate certain aspects of the present invention and to aid those of skilled in the art in practicing this invention. These Examples are in no way to be considered to limit the scope of the invention in any manner. Without further elaboration, it is believed that one skilled in the art can, based on the description herein, utilize the present invention fully.

Materials and Methods

PCR Amplification and Cloning of Nucleotides Encoding Fusion Proteins

Target genes for use in the expression of the CBD-intein-teriparatide fusion protein (SEQ ID NO: 9), CBD-intein-Spacer(S)-TEV site-teriparatide fusion protein (SEQ ID NO: 7), and CBD-intein-S-TEV site-liraglutide fusion protein (SEQ ID NO: 8; LX1) were respectively amplified with the primer pairs of SEQ ID NO: 10 and 11, SEQ ID NO: 12 and 13, and SEQ ID NO: 14 and 15 to incorporate Sap I and Pst I restriction sites by PCR method. Target genes for use in the expression of fusion protein containing 2 (LX2) or 3 copies (LX3) of CBD-intein-S-TEV site-liraglutide were amplified using the same protocol for expressing LX1.

The amplified product was then cloned into the Sap I to Pst I sites in the pTWIN1 expression vector (Source) using the IMPACT™ kit (NEB #E6901). pTWIN1 expression vector is a protein purification system that utilizes the inducible self-cleavage activity of protein splicing elements (i.e., intein) to separate the target protein from the affinity tag. For cloning into the pTWIN1 expression vector, the reaction mixture containing the amplified target gene fragment was purified per the manufacture's instruction. Then, the above purified gene fragment was double-digested with the restriction enzymes Sap I and Pst I in a reaction mixture. At the same time, a pTWIN vector was digested with the same enzymes in a reaction mixture. Following a 2 to 4-hour digestion, ligation was carried out per the manufacture's instruction.

Fusion Protein Expression

Transformation was done per the manufacturer's instruction. E. coli strain BL21(DE3) transformed to express the fusion protein was purified.

For cell culture, 1 liter of LB medium containing 100 μg/ml ampicillin was inoculated with a freshly grown colony or 10 ml of freshly grown culture. The culture was incubated in an air shaker at 37° C. until the OD₆₀₀ reaches 0.5-0.7. To induce the expression of the fusion protein, IPTG was added to a final concentration of 0.5 mM before the culture was transferred to a 15° C. air shaker overnight. The cells from the IPTG-induced culture were spun down at 5000×g for 15 minutes at 4° C. and the supernatant was discarded; cell pellet was stored at −20° C. for future use.

Affinity Purification

Before affinity purification, clarified cell extracts were prepared from the above cell pallets per the manufacturer's instruction. After chitin column equilibration, the clarified cell extracts were slowly loaded onto the chitin columns at a flow rate of no more than 0.5-1.0 ml/min. On-column cleavage was induced by flushing the column quickly with about three column volumes of Buffer B2 [20 mM HEPES or Tris-HCl, pH 6.5 containing 500 mM NaCl, and 1 mM EDTA]. Then, the column flow was stopped and the column was left at room temperature overnight.

Intein-Mediated Self-Cleavage of Fusion Protein

For on-column cleavage, the target protein was eluted using cleavage buffers having different pH values (pH 5.0, 6.0, and 6.7) per the manufacturer's instruction. 40 μl samples from each fraction was removed and respectively mixed with 20 μl 3×SDS Sample Buffer. The mixture was then centrifuged and the supernatant was discarded. Next, 40 μl of 1×SDS Sample Buffer was added to the pellet and boiled 5 minutes before electrophoresis. The remaining intein-tag and uncleaved fusion proteins were stripped from the column with 1% SDS. To assess cleavage efficiency, 200 μl chitin resin was removed and mixed with 100 μl 3×SDS Sample Buffer. After boiling for 5 minutes, the supernatant was analyzed on SDS-PAGE to determine the cleavage efficiency.

Intein-mediated self-cleavage of the fusion protein was also carried out without affinity purification. Briefly, clarified cell extracts were mixed with using cleavage buffers having different pH values (pH 5.0, 6.0, and 6.7). Cellular proteins from the mixture were separated by SDS-PAGE, and then transferred to PVDF membrane for Western blot detection with anti-GLP-1 (7-36) antibody for liraglitide precursor and anti-PTH antibody for teriparatide, followed by incubation with appropriate HRP-conjugated secondary antibodies.

Enzymatic Cleavage of Fusion Protein with TEV Protease

For enzymatic cleavage of the fusion protein, the fusion protein was incubated with TEV protease buffer (Lira fusion protein: TEV protease=50:1(mole)) for at least 16 hours.

Product Characterization

The teriparatide and liraglutide proteins were purified by high-performance liquid chromatography (HPLC) using Agilent 1260 Infinity II LC System with C18 column to 95% purity.

The identification of teriparatide and liraglutide proteins was carried out by mass spectrometry on MALDI TOF machine.

### Example 1

Intein-Mediated Self-Cleavage of CBD-Intein-Teriparatide Fusion Protein

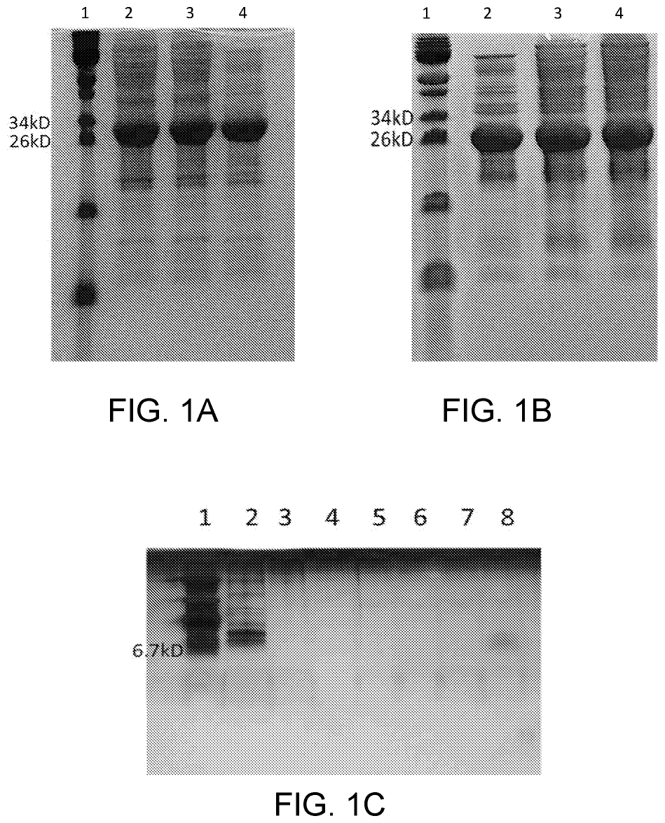

In this example, the freshly-prepared CBD-intein-teriparatide fusion protein was treated with cleavage buffers in various pH values and then analyzed by SDS-PAGE to investigate the intein-mediated self-cleavage of the fusion protein (FIG. 1A). Theoretical molecular weight of the CBD-intein-teriparatide fusion protein is 29,280.41 daltons (about 29 kDa), while the molecular weight of teriparatide is approximately 4.2 kDa. Referring to FIG. 1A, the CBD-intein-teriparatide fusion protein in lanes 2 to 4 corresponded to the broad band around 30 kDa (in comparison to the molecular weight marker in lane 1). This result indicated that there was no intein-mediated self-cleavage of the fusion protein at pH 6.7 (lane 2), pH 6.0 (lane 3) or pH 5.0 (lane 4). Generally, a cleavage buffer of pH 6.0-7.0 is sufficient to induce the intein-mediated self-cleavage, while a lower pH value often results in a more efficient self-cleavage. Accordingly, this SDS-PAGE result was quite unexpected.

In a further analysis, the CBD-intein-teriparatide fusion protein was treated with afore mentioned cleavage buffers at various pH values for a month to investigate whether a longer reaction period would facilitate the intein-mediated self-cleavage. The SDS-PAGE result, as provided in FIG. 1B indicated that even after a one-month reaction period, no intein-mediated self-cleavage was observed at pH 6.7 (lane 2), pH 6.0 (lane 3) or pH 5.0 (lane 4).

Cell lysates from reaction mixtures treated with the cleavage buffer (pH 6.7) for one month was subjected to affinity purification using a chitin column, followed by SDS-PAGE analysis. The result, as provided in FIG. 1C, indicated that there was no free teriparatide in the loaded lysate (lane 2), flow-through (lane 3), column wash (lane 4), or the first to fourth elusions (lanes 5-8, respectively).

Taken together, intein failed to effectuate the self-cleavage reaction in the CBD-intein-teriparatide peptide even when acidic conditions or a long treatment time was employed.

### Example 2

Enzymatic Cleavage of CBD-Intein-S-TEV Site-Teriparatide Fusion Protein with TEV Protease

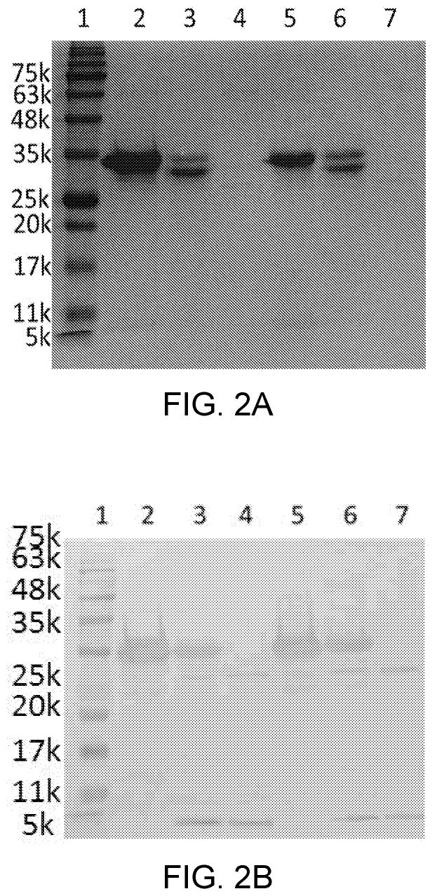

In view of the unexpected results of Example 1, a CBD-intein-spacer-TEV site-teriparatide fusion protein was prepared. In this novel platform, a TEV cleavage site was introduced, together with a spacer moiety, to facilitate a TEV protease-mediated cleavage. The spacer moiety was designed to have a flexible G-S linker with an N-terminal of proline for avoiding the self-cleavage resulted from intein. The CBD-intein-spacer-TEV site-teriparatide fusion protein was first purified, and then treated with TEV protease under different pH, before SDS-PAGE analysis (FIG. 2A) and western blot analysis (FIG. 2B).

Referring to both FIGS. 2A and 2B, in the purified products, a single broad band about 35 kDa was observed in lane 2 (pH 8.0) and in lane 5 (pH 7.0) (in relation to the molecular weight marker in lane 1), which indicated that there was no cleavage of the CBD-intein-spacer-TEV site-teriparatide fusion protein (theoretical molecular weight: 30,802.9 Da). In contrast, two bands (one near the 35 kDa marker, the other slightly below the former) were seen in the sample from the purified protein treated with TEV protease under pH 8.0 (lane 3) or pH 7.0 (lane 4), suggesting the CBD-intein-spacer-TEV site-teriparatide fusion protein was cleaved. Lanes 4 and 7 were respectively loaded with teriparatide in pH 8.0 and pH 7.0 buffer, and no cleavage was observed.

The data in FIGS. 2A and 2B indicated that cleavage by the TEV protease resulted in the release of teriparitide.

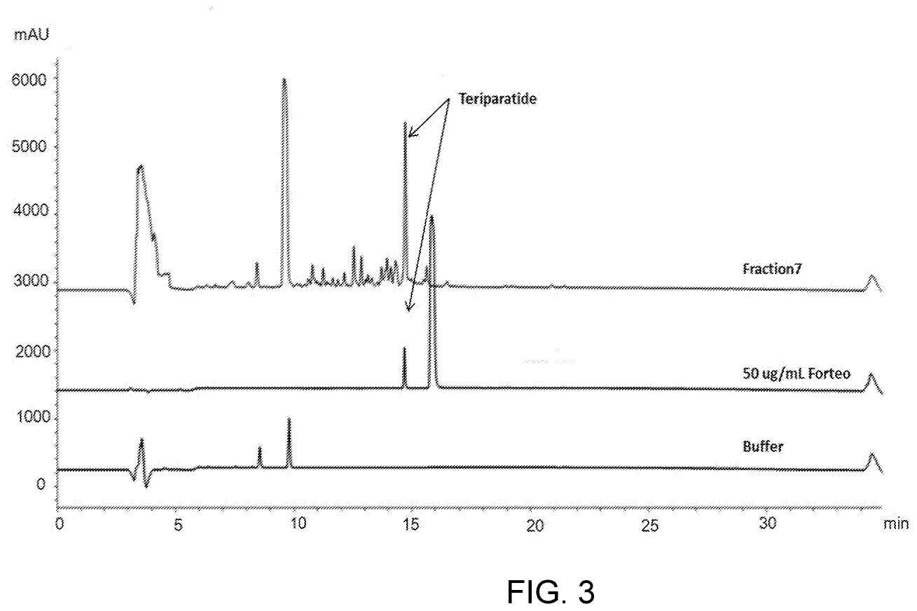

After the treatment with TEV protease, the reaction mixture was purified using HPLC, and the HPLC elution profile was provided in FIG. 3, which showed that the purified teriparatide had a similar elution retention time with respect to the reference sample. The thus-purified teriparitide was then characterized using the mass spectrometry, and the MS result indicated that the teriparitide had a molecular weight of 4,115.17 Da, which is very close to the theoretical molecular weight of 4,115.13 Da. These results indicated that the thus-obtained teriparitide is highly similar to the reference teriparitide at least in their primary structure.

### Example 3

Enzymatic Cleavage of CBD-Intein-S-TEV Site-Liraglutide Fusion Protein with TEV Protease

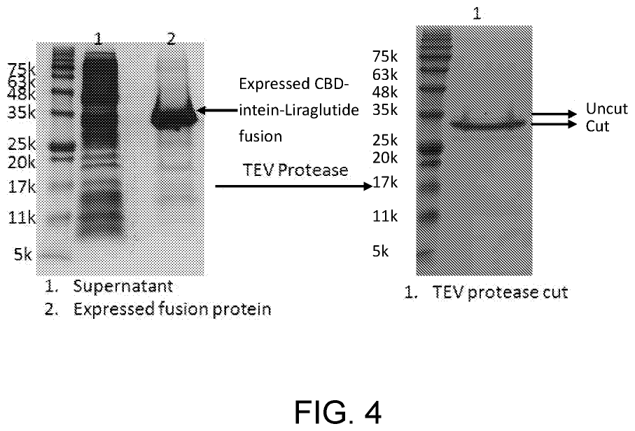

A fusion protein carrying liraglutide was prepared by use of the platform of Example 2. Accordingly, the CBD-intein-S-TEV site-liraglutide fusion protein was generated and subject to SDS-PAGE analysis. The supernatant (lane 2), as well as the insoluble fraction from the cell lysate (lane 3), was successfully separated using SDS-PAGE analysis (FIG. 4, left panel). In lane 3 of the left panel of FIG. 4, a single broad band below the 35 kDa marker is a clear indication of the expression of the CBD-intein-S-TEV site-liraglutide fusion protein (theoretical molecular weight: 30,068.9 Da). This band was excised and electroeluted to obtain the fusion protein, which was then treated with TEV protease (pH 8.0), and the product was analyzed using SDS-PAGE (FIG. 4, right panel). In lane 2 of the right panel, two bands were seen; the light band closer to the 35 kDa marker represented the un-cut CBD-intein-S-TEV site-liraglutide fusion protein, while the heavy band below represented the cleaved fusion protein. These data indicated that cleavage by the TEV protease resulted in the release of liraglutide.

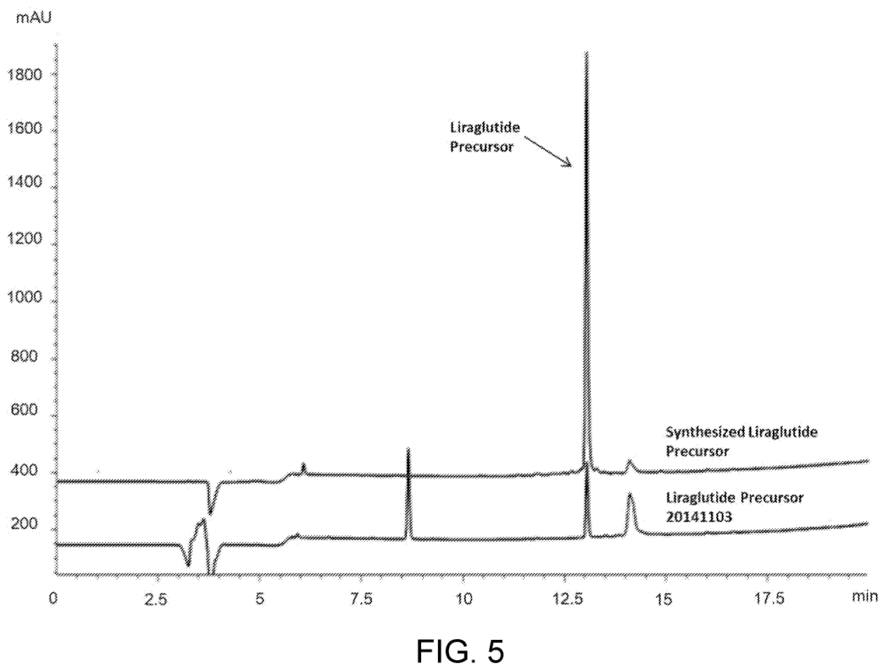

After the treatment with TEV protease, the reaction mixture was purified using HPLC, and the HPLC elution profile was provided in FIG. 5, which showed that purified liraglutide precursor had similar elution retention time with reference sample. The thus-purified liraglutide precursor was then characterized using the mass spectrometry, and the MS result indicated that the liraglutide precursor had a molecular weight of 3,382.6, which is very close to the theoretical molecular weight of 3,383.7. These results indicated that the thus-obtained liraglutide precursor is highly similar to the reference liraglutide precursor at least in their primary structure.

### Example 4

Expression of Multi-Copy CBD-Intein-S-TEV Site-Liraglutide Fusion Protein Using Different Expression Systems

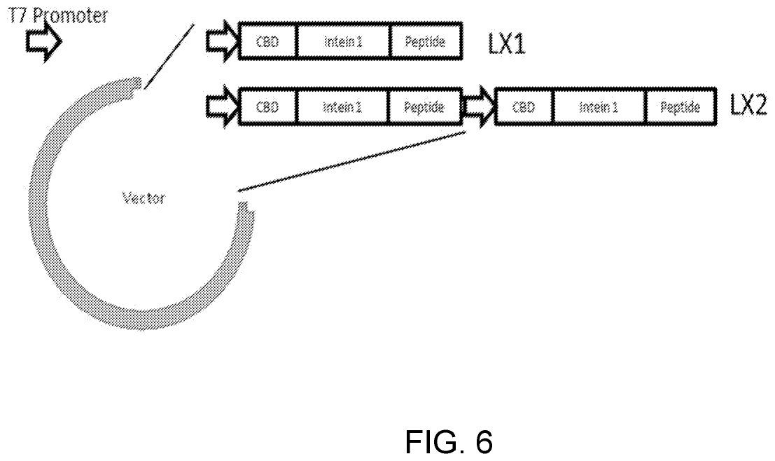

Vectors carrying one (LX1) or two copies (LX2) of CBD-intein-S-TEV site-liraglutide were constructed as illustrated in FIG. 6, using the protocol set forth above. The vector was then transformed into different bacterial strain.

(4.1) BL21(DE3) Strain

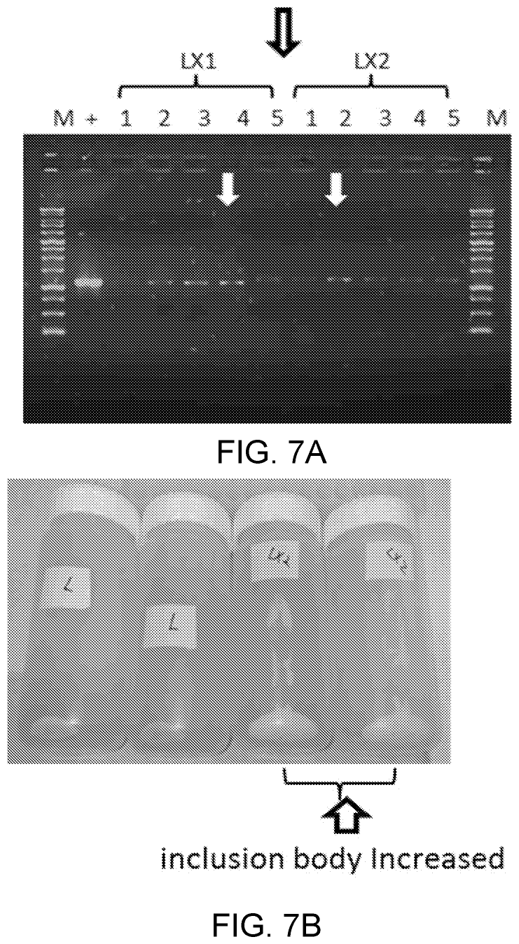

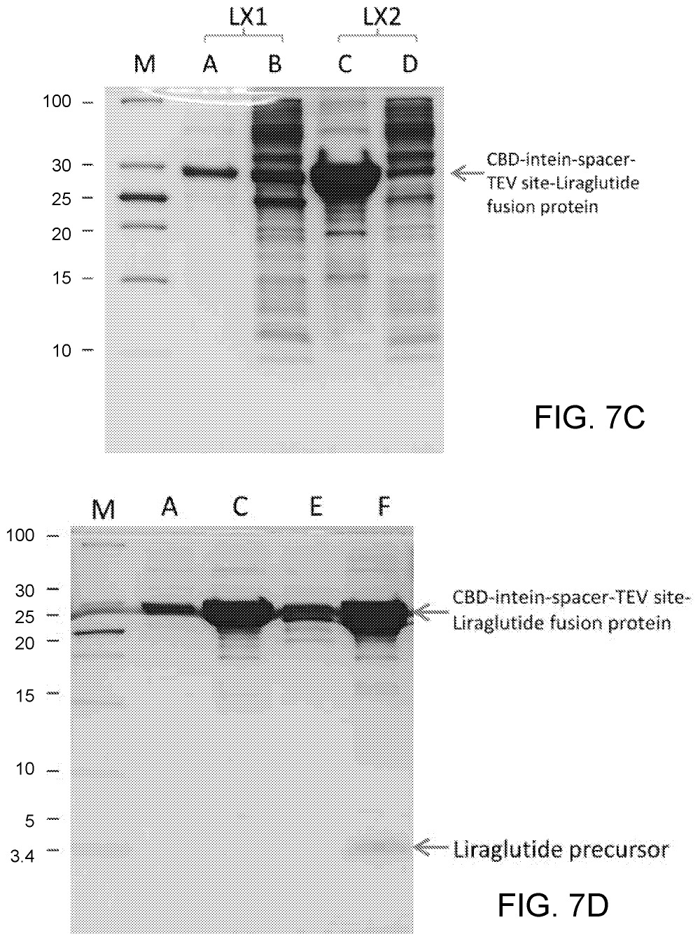

The transformed E. coli BL21(DE3) bacteria were grown as described above, and transformation was visualized by gel electrophoresis (FIG. 7A). The expressed protein was accumulated as insoluble aggregates in the inclusion bodies. The photograph of FIG. 7B indicates that the volume of the inclusion body substantially increased in bacteria transformed with the LX2 vector. Coomassie blue staining also indicates that the expressed protein (i.e., the CBD-intein-S-TEV site-liraglutide fusion protein) in the inclusion body increased substantially in bacteria transformed with the LX2 vector (FIG. 7C; M: marker, A: LX1 inclusion body; B: LX1 supernatant; C: LX2 inclusion body; D: LX2 supernatant).

The expressed fusion protein was digested with TEV, followed by HPLC purification. The results of coomassie blue staining, as summarized in FIG. 7D, and HPLC analysis (see, Table 1, below) indicate that the protein yield in bacteria transformed with the LX2 vector increased by at least 45 folds, as compared with bacteria transformed with the L1 vector (FIG. 7D; M: marker, A: LX1 inclusion body; C: LX2 inclusion body; E: LX1 after TEV enzymatic digestion; F: LX2 after TEV enzymatic digestion).

TABLE 1

| | Con. | Total |
|---|---|---|
| Sample | (mg/mL) | (mg) |
| LX1 | 0.011 | 0.176 |
| LX2 | 0.127 | 8.128 |

(4.2) T7 Expression Strain

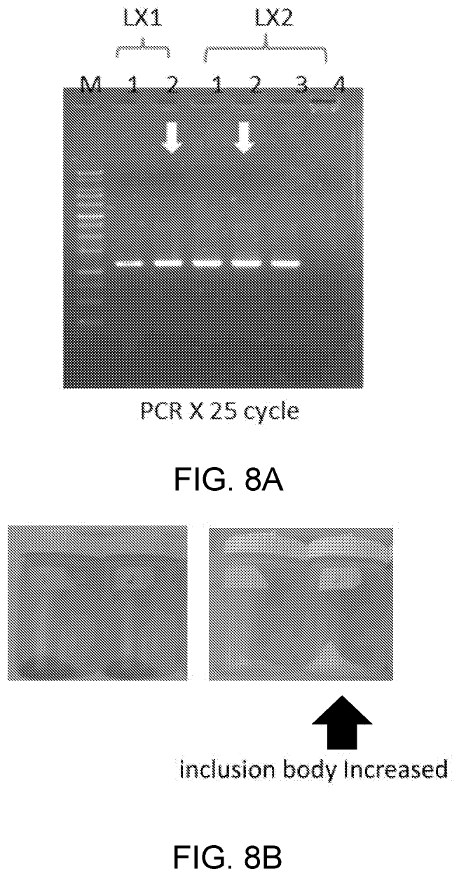

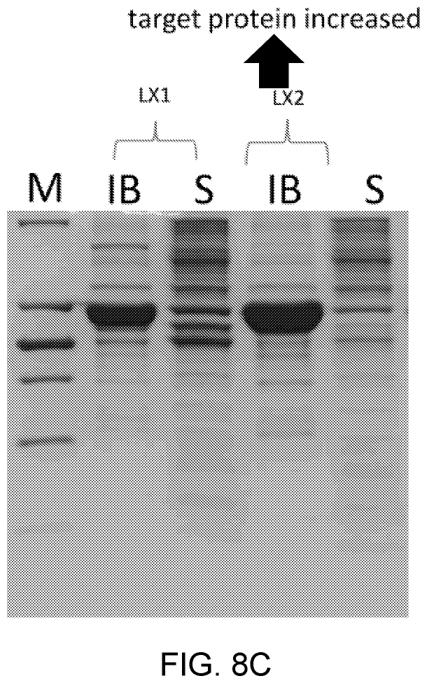

The transformed E. coli T7 expression bacteria were grown as described above, and transformation was visualized by gel electrophoresis (FIG. 8A). The expressed protein (cultured in 100 mL medium) was accumulated as insoluble aggregates in the inclusion bodies. The photograph of FIG. 8B indicates that the volume of the inclusion body substantially increased in bacteria transformed with the LX2 vector. Coomassie blue staining also indicates that the expressed protein (i.e., the CBD-intein-S-TEV site-liraglutide fusion protein) in the inclusion body increased substantially in bacteria transformed with the LX2 vector (FIG. 8C).

(4.3) Rosetta Expression Strain

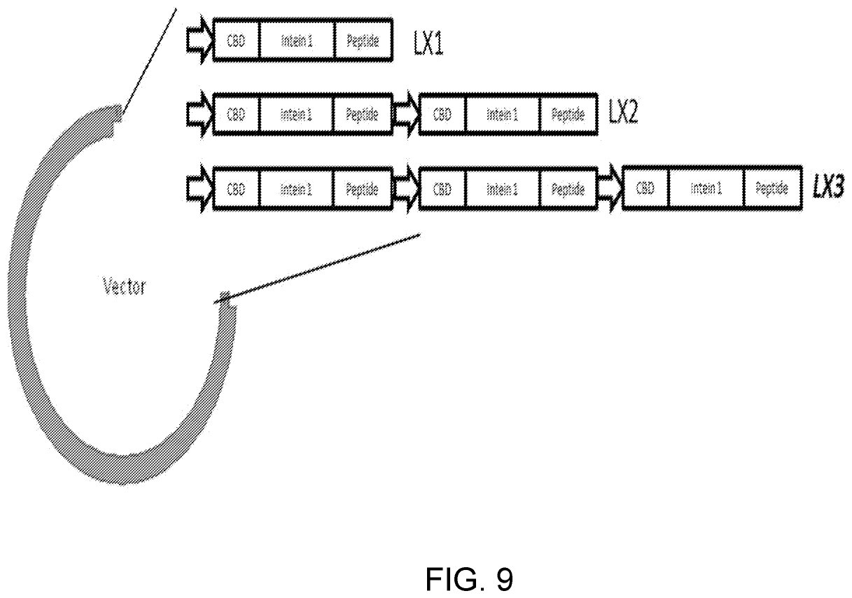

Vectors carrying one (LX1, or L), two (LX2) or 3 (LX3) copies of CBD-intein-S-TEV site-liraglutide were constructed as illustrated in FIG. 9, using the protocol set forth above. The vector was then transformed into different bacterial strain.

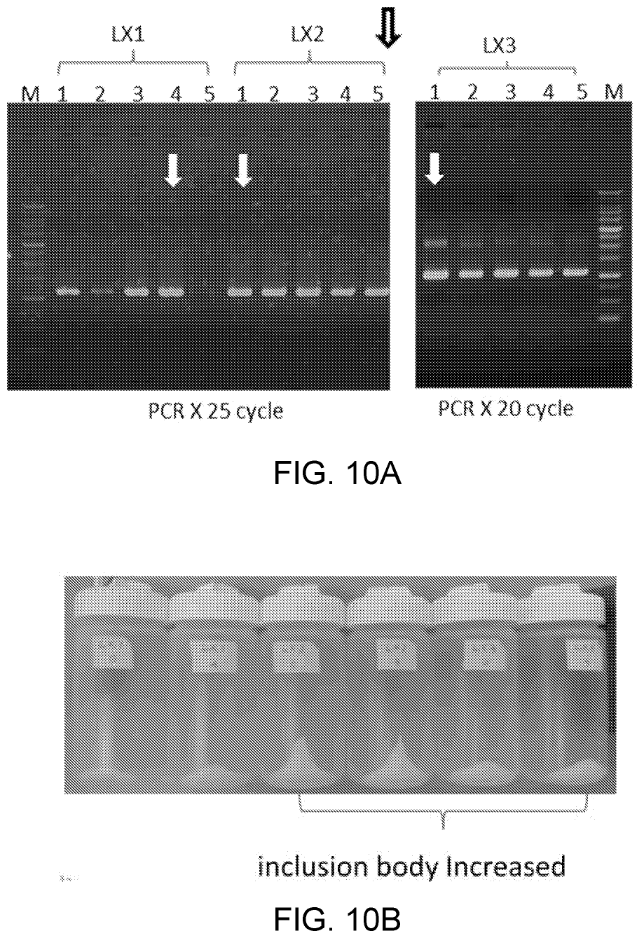

The transformed E. coli Rosetta expression bacteria were grown as described above, and transformation was visualized by gel electrophoresis (FIG. 10A). The expressed protein (cultured in 100 mL medium) was accumulated as insoluble aggregates in the inclusion bodies. The photograph of FIG. 10B indicates that the volume of the inclusion body substantially increased in bacteria transformed with the LX2 vector or LX3 vector, as compared with the LX1 vector.

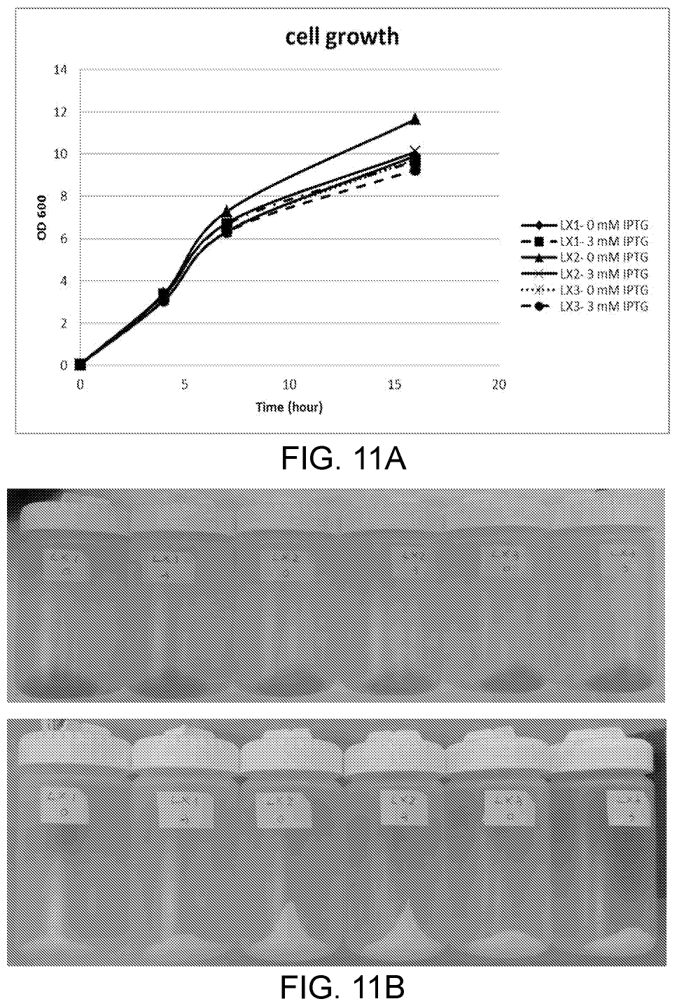

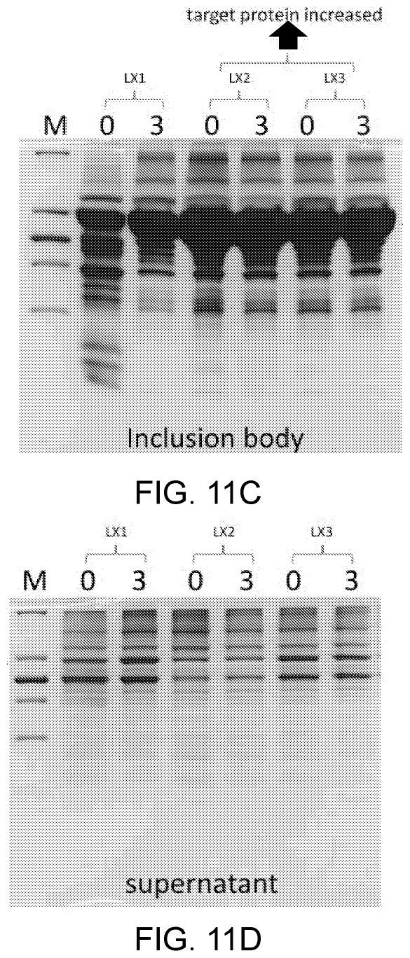

Growth rates of transformed bacteria under different conditions were summarized in FIG. 11A. The photograph of FIG. 11B indicates that the volume of the inclusion body substantially increased in bacteria transformed with the LX2 vector or LX3 vector, as compared with the LX1 vector. Coomassie blue staining results provided in FIG. 11C indicate that the expressed protein (i.e., the CBD-intein-S-TEV site-liraglutide fusion protein) in the inclusion body increased substantially in bacteria transformed with the LX2 vector or LX3 vector. On the other hand, there is no significant different regarding the amount of the expressed protein in the supernatants (FIG. 11D).

### Example 5

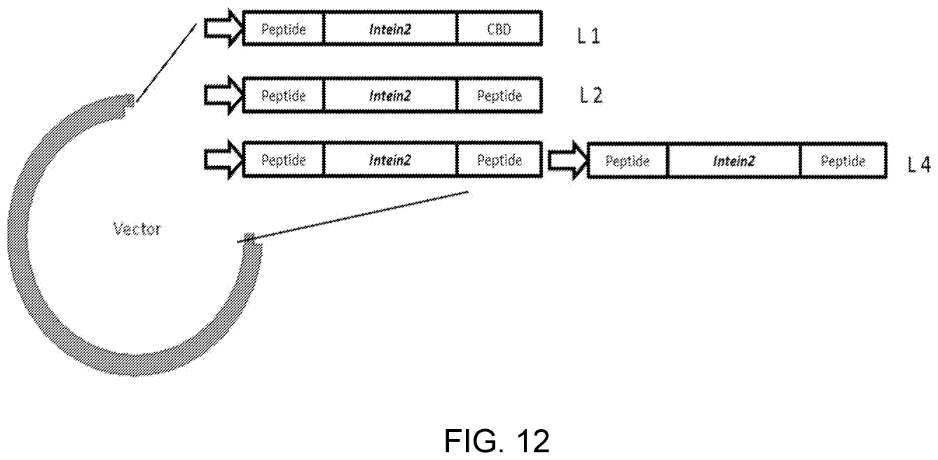

Target genes for use in the expression of fusion protein in this example were constructed as illustrated in FIG. 12. Briefly, the L1 vector comprises, sequentially, a T7 promoter, a liraglutide fragment, a self-cleavage peptide or catalytic cleavage protein (intein 2, SEQ ID NO: 17) and a CBD peptide; the L2 vector comprises one expression unit that comprises, sequentially, a T7 promoter, a first liraglutide fragment, an intein 2 fragment, and a second liraglutide fragment; and the L4 vector comprises two said expression units. The vector was then transformed into the KRX strain.

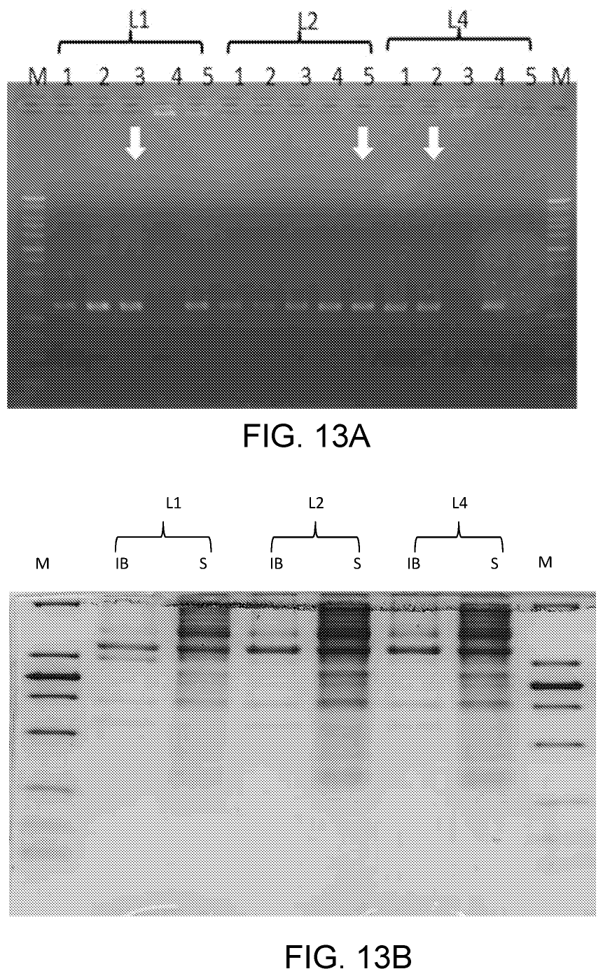

The transformed bacteria were grown as described above, and transformation was visualized by gel electrophoresis (FIG. 13A). Coomassie blue staining results of the expressed protein (cultured in 100 mL medium) provided in FIG. 13B indicate that the expressed protein in the inclusion body increased substantially in bacteria transformed with the L2 vector or L4 vector.

It will be understood that the above description of embodiments is given by way of example only and that various modifications may be made by those with ordinary skill in the art. The above specification, examples, and data provide a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of this invention.

1. An expression construct, comprising at least one expression unit, wherein the expression unit comprises a nucleotide sequence encoding a fusion protein, wherein the fusion protein comprises, sequentially from N-terminus to C-terminus, an affinity tag moiety, a spacer moiety, an enzymatic cleavage site, and a protein of interest moiety.

2. The expression construct of claim 1, wherein the expression construct comprises two or more said expression units.

3. The expression construct of claim 1, wherein the spacer moiety comprises the amino acid sequence of SEQ ID NO: 2.

4. The expression construct of claim 1, wherein the protein of interest moiety comprises the amino acid sequence of SEQ ID NO: 4, 5, 6, or 16.

5. (canceled)

6. The expression construct of claim 1, wherein the affinity tag moiety is a chitin binding domain (CBD)-intein.

7. The expression construct of claim 6, wherein the CBD-intein comprises the amino acid sequence of SEQ ID NO: 1.

8. The expression construct of claim 1, wherein the enzymatic cleavage site is a tobacco etch virus (TEV) cleavage recognition site.

9. The expression construct of claim 8, wherein the TEV cleavage recognition site comprises the amino acid sequence of SEQ ID NO: 3.

10. The expression construct of claim 1, wherein the fusion protein comprises the amino acid sequence of SEQ ID NO: 7 or 8.

11. The expression construct of claim 1, wherein each expression unit further comprises a promoter upstream to the nucleotide sequence encoding the fusion protein.

12. An expression construct, comprising at least one expression unit, wherein the expression unit comprises a nucleotide sequence encoding a fusion protein, wherein the fusion protein comprises two protein of interest moieties, and a self-cleavage peptide or a catalytic cleavage protein disposed between said two protein of interest moieties.

13. The expression construct of claim 12, wherein each expression unit further comprises a promoter upstream to the nucleotide sequence encoding the fusion protein.

14. The expression construct of claim 13, wherein the expression construct comprises two or more said expression units.

15. The expression construct of claim 13, wherein the self-cleavage peptide or the catalytic cleavage protein comprises the amino acid sequence of SEQ ID NO: 17.

16. The expression construct of claim 13, wherein each protein of interest moiety is selected from the group consisting of SEQ ID NO: 4, 5, 6, and 16.

17. A method for producing a protein of interest, comprising the steps of,
(a) providing an expression construct according to claim 1;
(b) transforming a host cell with the expression construct and culturing the host cell under conditions that allow the expression of the fusion protein;
(c) lysing the host cell to obtain a lysate comprising a soluble fraction and an insoluble fraction;
(d) optionally purifying the insoluble fraction to obtain a purified insoluble fraction;
(e) solubilizing the insoluble fraction from the step (d) or the purified insoluble fraction from the step (d) to obtain a solubilized fusion protein;
(f) suspending the solubilized fusion protein in a renaturation buffer thereby allowing the refolding of the solubilized fusion protein to obtain a refolded fusion protein; and
(g) cleaving the enzymatic cleavage site of the refolded fusion protein to release the protein of interest.

18. The method of claim 17, wherein the expression construct comprises two or more said expression units.

19. The method of claim 17, wherein the spacer moiety comprises the amino acid sequence of SEQ ID NO: 2.

20. The method of claim 17, wherein the protein of interest moiety comprises the amino acid sequence of SEQ ID NO: 4, 5, 6, or 16.

21. The method of claim 17, wherein the affinity tag moiety is a chitin binding domain (CBD)-intein.

22. The method of claim 21, wherein the CBD-intein comprises the amino acid sequence of SEQ ID NO: 1.

23. The method of claim 21, wherein the step (d) is performed using an affinity column that is specific to the affinity tag moiety affinity column.

24. The method of claim 23, wherein the affinity column is a chitin column.

25. The method of claim 17, wherein the enzymatic cleavage site is a tobacco etch virus (TEV) cleavage recognition site.

26. The method of claim 25, wherein the TEV cleavage recognition site comprises the amino acid sequence of SEQ ID NO: 3.

27. The method of any of claim 17, wherein the fusion protein comprises the amino acid sequence of SEQ ID NO: 7 or 8.

28. The method of any of claim 17, wherein the host cell is a prokaryotic cell.

29. The method of claim 28, wherein the prokaryotic cell is E. coli cell.

30. The method of claim 17, wherein each expression unit further comprises a promoter upstream to the nucleotide sequence encoding the fusion protein.
